(19) United States
(12) Patent Application Publication
(10) Pub. No.: US 2013/0344003 A1
(43) Pub. Date: Dec. 26, 2013

(54) IMMUNO-THERAPY FOR CANCER TREATMENT USING IRON OXIDE NANOPARTICLES

(71) Applicant: The Board of Trustees of the Leland Stanford Junior University, Palo Alto, CA (US)

(72) Inventor: Heike E. Daldrup-Link, San Francisco, CA (US)

(21) Appl. No.: 13/923,962

(22) Filed: Jun. 21, 2013

Related U.S. Application Data
(60) Provisional application No. 61/663,681, filed on Jun. 25, 2012.

(51) Int. Cl. A61K 33/26; A61K 49/08

(52) U.S. Cl. CPC A61K 33/26; A61K 49/08
USPC 424/9.36; 424/9.1

(57) ABSTRACT

An immuno-therapy for treatment of a tumor is provided. An effective dose of a composition containing a low dose of superparamagnetic iron oxide nanoparticle is administered to a tumor. Once the composition has been administered, it is recommended to avoid any means that would cause direct cytotoxic effects to the cancer cells and to normal/healthy tissue. The combination of composition-administered cancer cells with the avoidance of direct cytotoxic effects has been shown to be successful to inhibit the growth of the cancer cells or result in aptosis of the cancer cells. Additional dose(s) can be administered when it is determined that: (i) the tumor starts to grow and/or (ii) the remaining composition falls below a threshold. The immuno-therapy method is a safe, clinically applicable, ready-to-use theranostic approach for cancer patients who are unable to start chemoradiotherapy in a timely manner, i.e. an effective interim or adjunctive treatment for patients.

### CROSS-REFERENCE TO RELATED APPLICATIONS

This application claims priority from U.S. Provisional Patent Application 61/663,681 filed Jun. 25, 2012, which is incorporated herein by reference.

### STATEMENT OF GOVERNMENT SPONSORED SUPPORT

This invention was made with Government support under contract CA156124 awarded by National Institutes of Health. The Government has certain rights in this invention.

### FIELD OF THE INVENTION

The invention relates to immuno-therapy for tumor treatment.

### BACKGROUND OF THE INVENTION

Complete surgical resection and combined chemoradiation represent the hallmarks for curative treatment of many cancer cancer patients. However, complete resection cannot always be achieved, especially for large tumors and tumors close to anatomical structures which cannot be removed (e.g. large vessels, nerves, and central nervous system). In addition, loco-regional recurrence remains a significant problem and adversely affects overall survival.

To eradicate residual tumor cells local radiation or combined chemoradiation therapy is recommended to be initiated within 8 weeks of surgery. However, patient compliance is limited with about a quarter of the patients choosing to omit or delay post-operative chemo-radiation. In addition, clinical, psychological or social factors can lead to prolonged delays in chemoradiation treatment initiation ranging from 3 months to 1 year after surgery.

Omission or significant delay of postsurgical radiotherapy and/or chemotherapy significantly reduces overall survival. Accordingly, it is desired to advance the art with new, safe, effective and easy-to-apply treatment options that can bridge the gap between surgery and adjuvant chemo-radiation to suppress tumor growth in the interim time period and ultimately, improve patient survival. The present invention bridges this gap.

### SUMMARY OF THE INVENTION

An immuno-therapy for treatment of a tumor is provided. An effective dose of a pharmaceutically accepted composition is administered in vivo to cancer cells of a tumor. The composition contains superparamagnetic iron oxide nanoparticles. Examples of useful nanoparticles are ferumoxytol, ferumoxtran-10 or ferumoxides. In one variation, the compositions could be chemically modified to attract or activate immune cells (such as macrophages or T-cells).

The effective dose is defined as: (i) 1-50 mg Fe/kg body weight and/or (ii) 1-10 mg Fe/ml of an administered iron product concentration. These doses, as described herein, are considered low doses and they do not cause (direct) cytotoxic effects to the cancer or normal/healthy cells.

Once the composition has been administered and during the immuno-therapy period, it is recommended to avoid any means that would cause direct cytotoxic effects to the cancer cells and to normal/healthy tissue. Examples on how such cytotoxic effects could be achieved are, for example, but not limited to: (i) heat applied to the composition-administered cancer cells, (ii) irradiation energy applied to the composition-administered cancer cells, (iii) a release of a toxic agent by the administered composition or to the administered composition, or (iv) any combination of these examples.

The combination of composition-administered cancer cells with the avoidance of direct cytotoxic effects during the period of the immuno-therapy has been shown to be successful to inhibit the growth of the cancer cells or to result in aptosis of the cancer cells.

Progress of the immuno-therapy can be evaluated using Magnetic Resonance Imaging (MRI, while the therapy is ongoing) to image: (i) the tumor to determine a size of the tumor, and/or (ii) the composition-administered to the cancer cells to determine the amount of the composition remaining in an enviroment of the cancer cells. It is noted that MRI used for these purposes does not cause direct cytotoxic effects to the cancer cells and to normal/healthy tissue. The imaging steps could be performed in a single imaging procedure or different imaging procedures. The determination of (i) and (ii) can be performed semi- or fully automatic by computer software either in conjunction with an MR imaging system or as part of an MR imaging system.

When it is determined that: (i) the tumor starts to grow and/or (ii) the remaining composition falls below a threshold, then an additional effective dose of the pharmaceutically accepted composition can be administered in vivo to the cancer cells of the tumor or the remaining cancer cells of the tumor. These additional doses falls in the same ranges as described supra.

The immuno-therapy of this invention is a safe (due to the lack of any concomitant local or systemic toxic side effects), clinically applicable, ready-to-use theranostic approach for cancer patients who are unable to start chemoradiotherapy in a timely manner, i.e. an effective interim or adjunctive treatment for patients. The immuno-therapy bridges the gap between surgery and adjuvant chemo-radiation to suppress tumor growth in the interim time period and ultimately, improve patient survival.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the immuno-therapy for tumor treatment method according to an exemplary embodiment of the invention.

FIG. 2 shows according to an exemplary embodiment of the invention iron oxide nanoparticles inhibition of tumor growth. Shown is a photo of a mouse at 16 days after co-implantation of 1.3 million PyMT-MMTV cancer cells with ferumoxytol (USPIO) or cancer cells only (control).

FIG. 3 shows an intra-individual comparison according to an exemplary embodiment of the invention. Decreased tumor volume of ferumoxytol-treated and untreated tumors at different time points after tumor inocculation. Data are displayed as means and standard deviations of n=10 tumors for the 10 mg Fe/kg dose group and controls (cancer-UL) as well as n=7 tumors for the 29.7 mg Fe/kg dose group and controls (cancer).

FIG. 4 shows an inter-individual comparison according to an exemplary embodiment of the invention. Decreased tumor volume of ferumoxtran-10-treated and untreated tumors at different time points after tumor inocculation. Data are displayed as means and standard deviations of n=6 tumors and controls.

FIG. 5 is an example indicating that ultrasmall paramagnetic iron oxide nanoparticles (USPIO) metabolization disinhibits tumor growth. FIG. 5 shows according to an exemplary embodiment of the invention in vivo MR imaging of iron oxide nanoparticles. Axial T2-weighted MR images of representative MMTV-PyMT mammary tumors at different time points after co-implantation of cancer cells and ferumoxytol (white arrow) and cancer cells only (red arrow). The iron oxide nanoparticle-based contrast agents cause a negative (dark) signal effect on these scans (white arrow).

FIG. 6 is another example indicating that ultra-small paramagnetic iron oxide nanoparticles (USPIO) metabolization disinhibits tumor growth. FIG. 6 shows according to an exemplary embodiment of the invention quantitation of iron-induced MR signal (T2*relaxation rates) of coimplantation sites and muscle as internal control up to 21 days after inoculation. Data are displayed as means of 10 implantation sites and standard deviations. *indicates significant difference (p<0.5).

FIG. 7 shows according to an exemplary embodiment of the invention that low dose (low as defined herein) ferumoxytol does not induce cytotoxic effects on cancer cells. Relative caspase 3/7 activities, assessed by AMC Caspase-3/7 assay, demonstrate no significant toxic effect on a variety of cell lines up to an exposure with 8.37 mg Fe/ml ferumoxytol. Further increase in ferumoxytol concentrations induced a mild, dose-dependent cytotoxic effect. Data were collected from 3 independent experiments per cell line.

FIG. 8 shows according to an exemplary embodiment of the invention USPIO increased macrophage migration. Macrophage-cancer cell co-culture set up in transwell chambers. DiD-labeled macrophages were seeded onto the insert and cancer cells were seeded onto the bottom chamber. Ferumoxytol was added to the lower chamber. Macrophage migration was analyzed 6 hours after addition of ferumoxytol at 2.73 mg/ml (equivalent to 10 mg Fe/kg used in in vivo studies calculated from the average animal weight) or PBS to the coculture system.

FIG. 9 shows according to an exemplary embodiment of the invention corresponding macrophage counts in the lower chamber, averaged from counts in 15-20 fields at 10×.

FIG. 10 shows according to an exemplary embodiment of the invention representative fluorescence images at 10× magnification demonstrate migration of more DiD-positive (red) macrophages towards lower chambers with cancer cells and ferumoxytol than chambers with cancer cells or ferumoxytol alone. Cell nuclei are counterstained with DAPI.

FIG. 11 shows according to an exemplary embodiment of the invention USPIO induced macrophage-mediated cancer cell apoptosis. Representative F-actin/rhodamine (red) and DAPI stains (upper row) as well as Caspase-3 immunostains (green, lower row) of cells in transwell coculture system (red). Cancer cells, incubated with both macrophages and ferumoxytol induce higher number of apoptotic cells than cancer cells incubated with macrophages or ferumoxytol alone.

FIG. 12 shows according to an exemplary embodiment of the invention USPIO upregulated M1-associated gene expression and downregulated M2-associated gene expression. Gene expression profiles of macrophages, incubated with cancer cells, ferumoxytol or both, measured by qRT-PCR. Data were collected from 3 independent experiments with triplicates each.

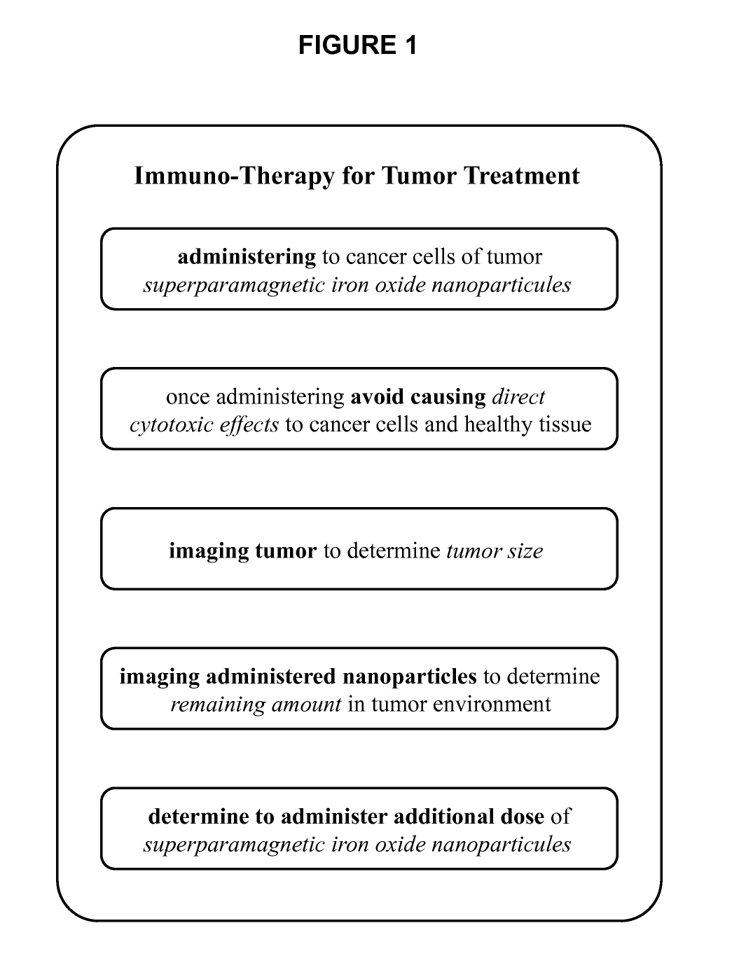

### DETAILED DESCRIPTION

### Therapeutic Effect of Iron Oxide Nanoparticles

In our experiments for this invention we found that tumor cells co-injected with superparamagnetic iron oxide nanoparticles showed a markedly delayed growth rate compared to tumor cells injected without the addition of iron oxides. The applied iron dose was too low to exert any direct toxic effect on adjacent cancer cells or normal/healhty tissue.

Iron oxide nanoparticles are internalized by macrophages in cancer. There are two primary macrophage phenotypes in the tumor microenvironment, namely pro-inflammatory M1 macrophages, which support rejection of developing cancers, and anti-inflammatory M2 macrophages, which stimulate tumor growth.

The phagocytosis of iron products modifies macrophage polarization and function. In vitro studies showed that relatively low doses of iron oxide nanoparticles (100 μg Fe/mL) can lead to induction of typical features of pro-inflammatory M1 macrophages, such as increased macrophage migration and production of inflammatory mediators, including tumor necrosis factor alpha (TNF-α), and nitric oxide (NO).

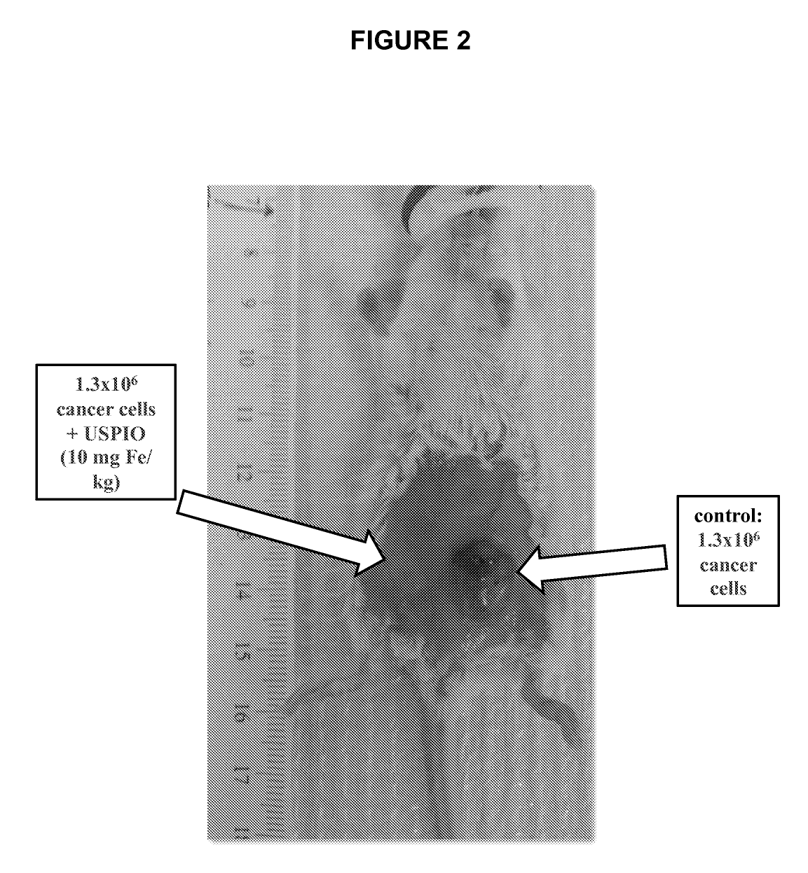

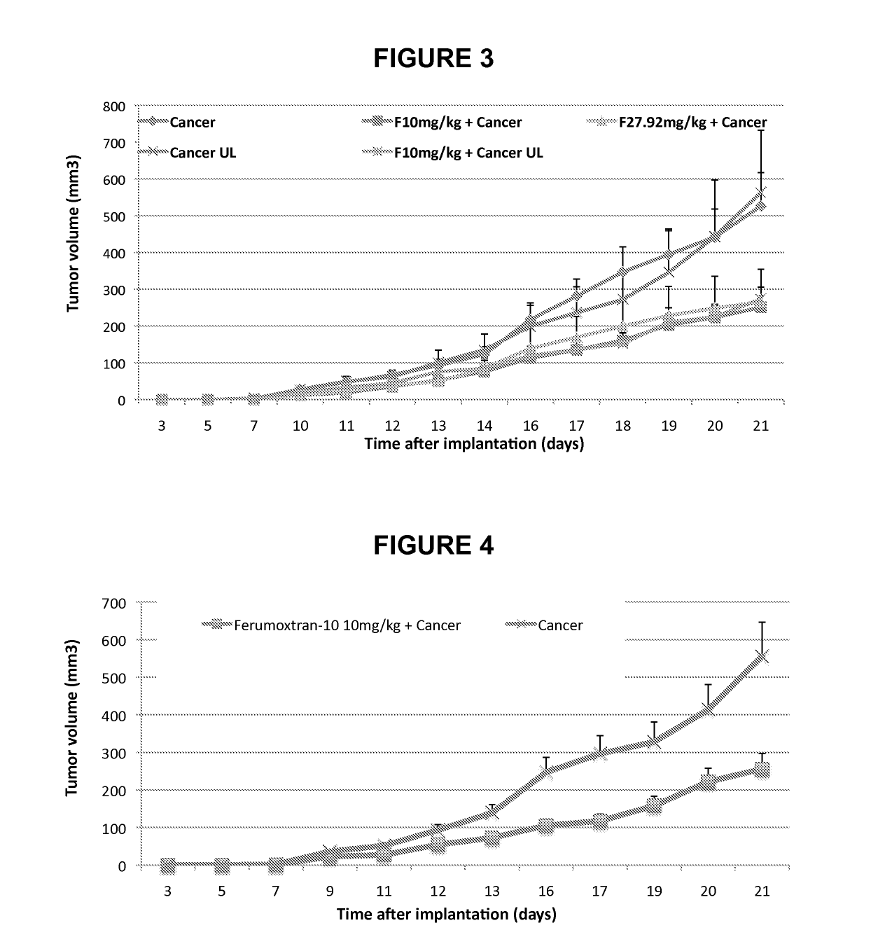

We developed an immuno-therapy encompassing the local administration of superparamagnetic iron oxide nanoparticles into (early stage) cancers which would attract reticuloendothelial macrophages, induce an M1 polarization, lead to secretion of pro-inflammatory cytokines, promote cancer cell death and thereby, inhibit overall tumor growth (FIGS. 2-4). This indirect therapeutic effect of superparamagnetic iron oxide nanoparticles would be advantageous compared to local administration of chemotherapies due to the lack of concomitant local tissue toxicity. In addition, the method does not include irradiation (i.e. such radiation is absent) of the injected superparamagnetic iron oxide nanoparticles. Such irradiation directed to the superparamagnetic iron oxide nanoparticles would generate thermal energy by the particles, whereby the particles enter the cytoplasm of the target cell and reduces proliferation of the target cells. Generally, the method does not include any application that results in cytotoxic effects on cancer cells and normal or healthy tissue as a result of (i) heat applied to the nanoparticles-administered cancer cells, (ii) irradiation energy applied to nanoparticles-administered cancer cells, (iii) a release of a toxic agent by the nanoparticles-administered or to the nanoparticles-administered, or (iv) any combination thereof.

The immuno-therapy method of this invention is a safe, clinically applicable, ready-to-use theranostic approach for cancer patients who are unable to start chemoradiotherapy in a timely manner. Our therapeutic strategy could provide a safe and effective interim or adjunctive treatment for these patients. The following description provides experimental data supporting the immuno-therapeutic method.

### Experimental Methods

### Contrast Agents

Two ultrasmall superparamagnetic iron oxide nanoparticle compounds (USPIO) were investigated:

1) Ferumoxytol (Feraheme, AMAG Pharmaceuticals Inc.) is a USPIO nanoparticle recently FDA approved for intravenous treatment of iron deficiency in patients with impaired renal function. Ferumoxytol has an iron oxide core and a carboxydextran coating. Ferumoxytol has a mean hydrodynamic diameter of 30 nm, an r₁ relaxivity of 38 s⁻¹ mM⁻¹ and an r₂ relaxivity of 83 s⁻¹ mM⁻¹ at 40 Mhz and at 37° C.

2). Ferumoxtran-10 (Sinerem, Guerbet, Paris, France) is a USPIO compound which had been previously investigated in clinical trials in Europe. Ferumoxtran-10 has a hydrodynamic diameter of 15-40 nm, an r1 of 22.7 s⁻¹ mM⁻¹ and an r2 relaxivity of 53 s⁻¹ mM⁻¹ at 0.47 T and 37° C., 20 Hz.

### In Vivo Evaluations of the Effect of Iron Oxide Nanoparticles on Tumor Growth

### Animal Model

Experiments were carried out in thirty-seven postpubertal female FVB/n mice (10-12 weeks), were randomly divided into the following experimental groups: Twenty four mice received injections of 1.2 million MMTV PyMT-derived tumor cells immersed in 10 mg Fe/kg ferumoxytol (n=10), 27.92 mg Fe/kg ferumoxytol (n=7) or 10 mg Fe/kg ferumoxtran-10 (n=6), into the right lower mammary fat pad as well as injections of 1.2 million MMTV PyMT-derived tumor cells only into the left lower mammary fat pad. Tumor size was measured with a caliper every other day. To address potential cross-talk of two tumors in the same mouse, 14 additional mice were implanted unilaterally with either 1.2 million MMTV-PyMT-derived cancer cells plus 10 mg/kg ferumoxytol (n=7) or cancer cells only (n=7) into the left mammary fat pads. In all groups, tumor sizes were measured with a caliper and cancer growth was calculated up to 21 days after implantation.

### MR Imaging of Ferumoxytol-Treated and Untreated Cancers

All animals underwent MR imaging under isofluorane anesthesia, using a 7 Tesla animal MR scanner (General Electric-Varian “microSigna 7.0”) and a dedicated single-channel transmit/receive birdcage radiofrequency coil (inner diameter of 2 cm). MR images were obtained at day using a 7 Tesla animal MR scanner (General Electric-Varian “microSigna 7.0”) and a 45 mm Millipede coil (Varian Inc., Palo Alto, Calif., USA). MR images were obtained at 2, 4, 7, 10, 14, and 21 days post-inoculation, using a pulse sequence of T2-weighted 2D fast gradient echo (FGRE) with repetition time 70 ms/echo times 1.5-12.6 ms (8 echoes with echo spacing of 1.6 ms)/flip angle 20°/matrix 128×128 pixels/field of view 4.5×2.7 cm/number of excitations 1/slice thickness 0.6 mm.

MR data was analyzed using custom research software tool (Cinetool, GE Global Research Center). T₂*relaxation times of tumors were calculated based on multi-echo FGRE images, converted to relaxation rates (R₂*=1/T₂*) and compared between MRI scans at different time points after tumor cell implantation.

### In Vitro Studies

### Caspase 3/7 Assay

To evaluate possible causes of iron-mediated suppression of cancer growth, we first evaluated potential direct toxic effects of iron oxides on various cell lines: RAW624.7 macrophages, HT1080, M D A-MB-468 and MDA-MB-435 cancer cells (ATCC) and MMTV-PyMT cells (isolated from 95 day old MMTV mice), human fibroblasts (ATCC) and human umbilical vein endothelial cells (HUVEC, Lonza, Clonetics, Walkersville, Md., USA) were incubated with increasing concentrations of ferumoxytol from 0-30 mg/ml for 6 hours and evaluated for intracellular caspase 3/7 activities using the SensoLyte Homogeneous AMC Caspase-3/7 assay kit (AnaSpec, Inc., Fremont, Calif., USA). Briefly, AMC caspase substrate solution was incubated with ferumoxytol-exposed cells and untreated controls at room temperature for 30 minutes. The fluorescence signal of the cell samples was measured by a fluorescence microplate reader (FlexStation 11384, Molecular Device, CA) with Ex/Em 354 nm/442 nm, cutoff 430 nm.

### Macrophage Migration Assay

We evaluated the migration of macrophages to ferumoxytol-immersed or untreated cancer cells in a transwell coculture system with 3 μm microporous membranes that permit cell translocation between chambers (Corning). Bone marrow macrophages were derived from femurs of MMTV PyMT mice and labeled with the lipophilic carbocyanine dye DiD (Interchim, Montlucon, France), using established techniques. Labeled macrophages were plated to the transwell inserts and MMTV-PyMT cancer cells were seeded to the bottom wells of dual chamber transwell plates, with or without addition of ferumoxytol to the lower chamber at 2.73 mg/mL. Of note, the high density of iron oxide nanoparticles prevents any major diffusion into higher chambers. Control groups were set up without either adding cancer cells, ferumoxytol or both in the co-culture system. After 6 hours of co-incubation, the bottom chambers were isolated and cells were stained by 4′,6-diamidino-2-phenylindole (DAPI, Invitrogen). Fluorescent macrophages that had migrated to the bottom chamber of transwelll systems were counted under a Zeiss fluorescence microscope (Zeiss, Oberkochen, Germany) with DAPI and DiD channels, using 1015 randomly selected fields at ×10 magnification. To evaluate iron and macrophage induced apoptosis, cancer cells were then stained against Human Active Caspase-3 Antibody (R&D System, Minneapolis, Minn., USA) at 1:100 dilutions in PBS, supplemented with 0.5% BSA. Counterstains of intracellular actins and nucleus were performed by incubating the cell samples for 1 hour with Rhodamine-Phalloidin (Invitrogen, Eugene, Oreg., USA) at 1:200 dilutions in 0.5% BSA containing PBS solution and fixed with a DAPI mounting solution (Invitrogen).

### M1/M2 Polarization Assays

To measure M1 and M2-associated gene expression in vitro, bone marrow derived macrophages, co-cultured with ferumoxytol and/or MMTV-PyMT cancer cells as described above, were collected, total RNA was extracted using the RNeasy mini/micro kit (Qiagen, Valencia, Calif., USA) following the manufacturer's protocol and 1 μg of total RNA was reverse-transcribed into complementary DNAs with an iScript complementary DNA synthesis kit (Bio-Rad, Hercules, Calif., USA) containing a mixture of oligo (dT) and random primers. Real-time PCR was performed with primers on an ABI PRISM 7900HT Sequence Detection System (Applied Biosystems, Foster City, Calif., USA), using a DyNAmo HS SYBR Green qPCR kit (New England BioLabs, Finnzymes, Finland). Cycling conditions were the following: initial denaturation at 95° C. for 10 min, followed by 40 cycles at 95° C. for 15 s and 60° C. for 1 min. mRNA expression levels were determined by a comparative Ct method.

### Results

### In Vivo Studies: Iron Oxide Nanoparticles Inhibit Tumor Growth

Co-injection of ultrasmall superparamagnetic iron oxide nanoparticles (USPIO) with MMTV PyMT cancer cells lead to significant inhibition of tumor growth when compared to tumor cells that were not exposed to iron oxides (FIGS. 2-4, p<0.05). Tumors derived from co-implantation of MMTV-PyMT cells and USPIO showed an average reduction of tumor volumes by 38-60% compared to control tumors that were not exposed to USPIO. There was no difference in tumor growth inhibition by two different USPIO doses, 10 and 27.9 mg Fe/kg (p>0.05) or two different USPIO compounds, ferumoxytol and ferumoxtran-10 (p>0.05). In addition, tumor volumes in mice inoculated unilaterally or inoculated bilaterally were not significantly different for either experimental group (p>0.05).

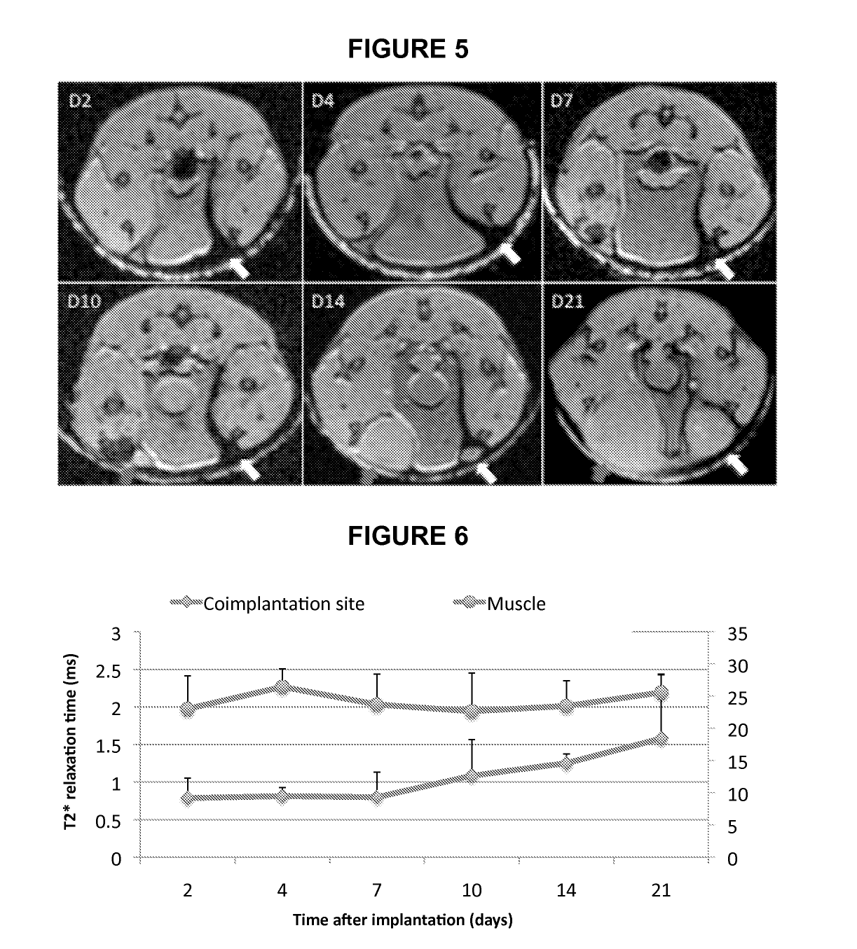

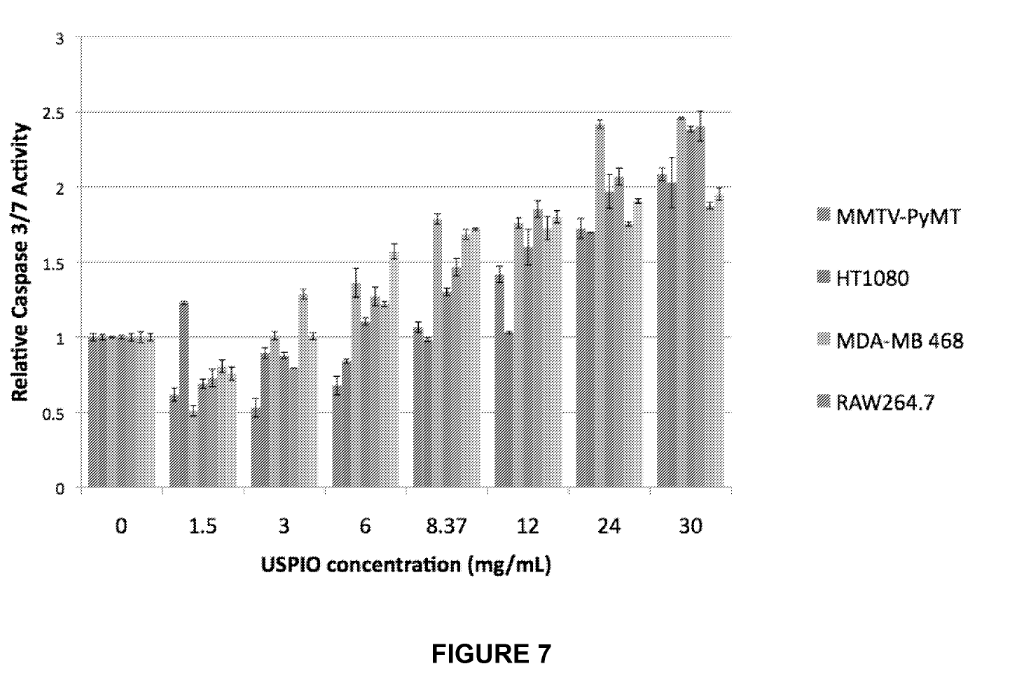

Serial MR images confirmed significant inhibition in tumor growth for MMTV PyMT cancer cells co-injected with iron oxides (p<0.05). Local iron deposition at the cancer cell transplant site could be visualized by significant darkening effect on T2-weighted MR images (FIG. 5). The r2*relaxation time at the site of tumor implantation, which is an indirect measure of local iron concentration, was significantly lower at cancer cell+ferumoxytol implantation sites compared to cancer cell only transplantation sites and muscle as an internal control (p<0.05).

This change in r2*relaxation time slowly decreased over about two weeks (FIG. 6). The disappearance of T2*signal effects at the cancer cell transplant site over time correlated with tumor-growth.

### In Vitro Studies: Iron Oxide Nanoparticles Induce Macrophage-Mediated Cancer Cell Death

Caspase 3/7 assay

To evaluate if our applied relatively low concentrations of dextran- and carboxydetran-coated iron oxide nanoparticles cause any direct toxic effects, we measured cancer cell apoptosis after incubation with ferumoxytol. We found no significant direct cytotoxic effects of ferumoxytol doses of 0-9 mg Fe/ml on a variety of cancer cell lines, fibroblasts and endothelial cells (p>0.05). Of note, a concentration of 2.73 mg Fe/mL ferumoxytol corresponds to 10 mg/kg used in in vivo studies. Further increase of ferumoxtol doses up to 30 mg/mL produced a minor dose-dependent cytotoxicity in these cell lines (p<0.05).

### Macrophage Migration Assay

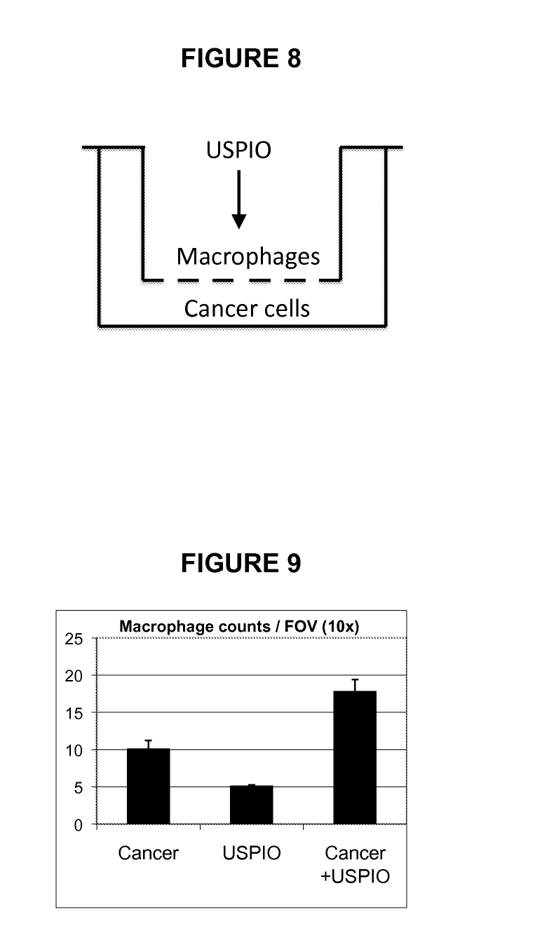

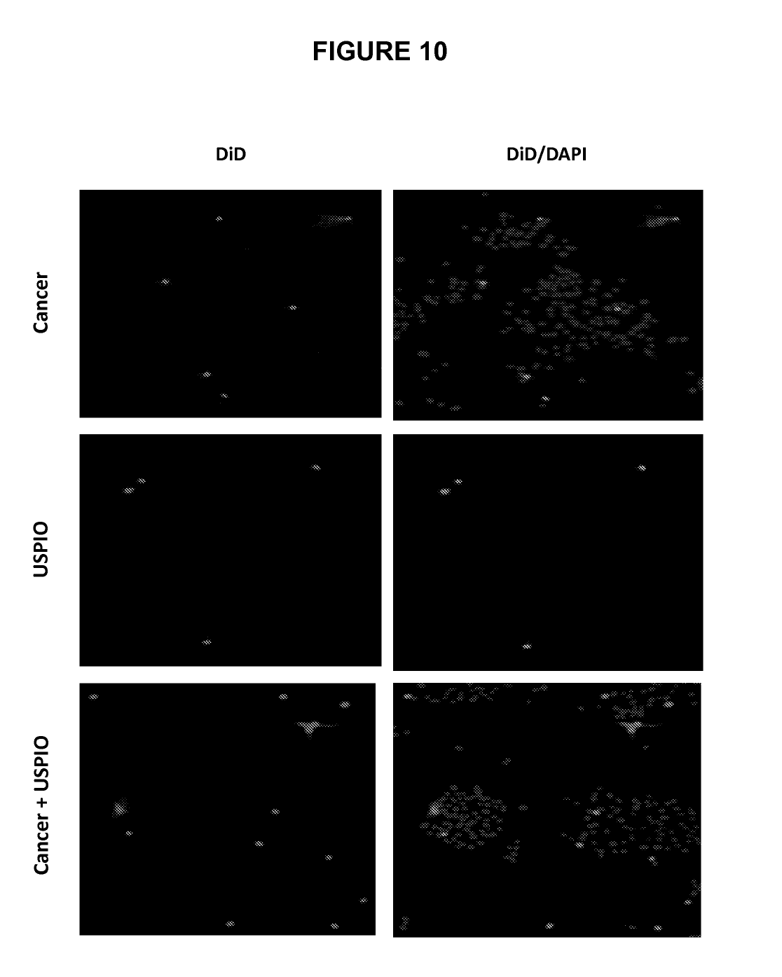

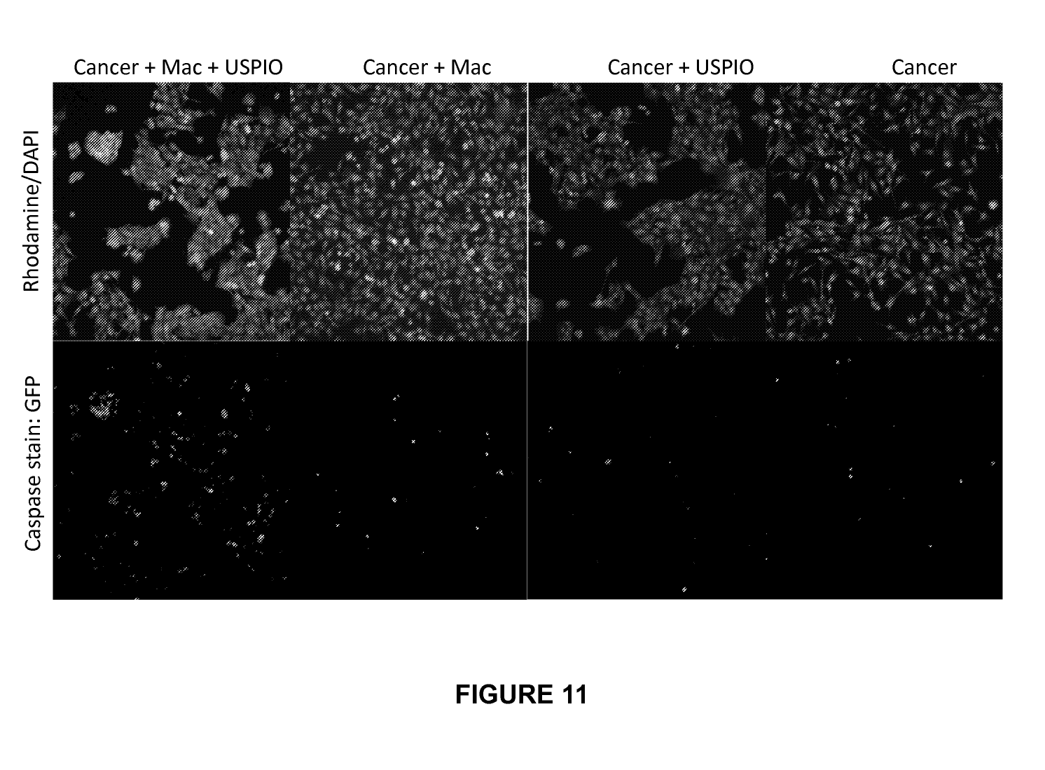

We investigated the effect of USPIO on macrophage migration to cancer cells in a dual chamber transwell system (FIG. 8). Ferumoxytol or cancer cells alone attracted migration of few macrophages (FIGS. 9-10). When both ferumoxytol and cancer cells were present in the lower chamber of the coculture system, a significantly increased macrophage migration towards cancer cells was noted by ˜2 folds at 6 hours after incubation (p<0.05).

Immunocytochemistry showed that 24 hours after ferumoxytol incubation at 2.73 mg/mL, there was increased cleaved caspase 3 expression in MMTV-PyMT cancer cells that had been co-incubated with ferumoxytol and macrophages, compared with cancer cells incubated with ferumoxytol or macrophages alone. Ferumoxytol treatment alone did cause significant activation of cleaved caspase 3 expression in cancer cells, indicating that low dose (as defined herein) ferumoxytol activated cancer cell apoptosis via macrophage-mediated pathways.

### M1/M2 Polarization Assays

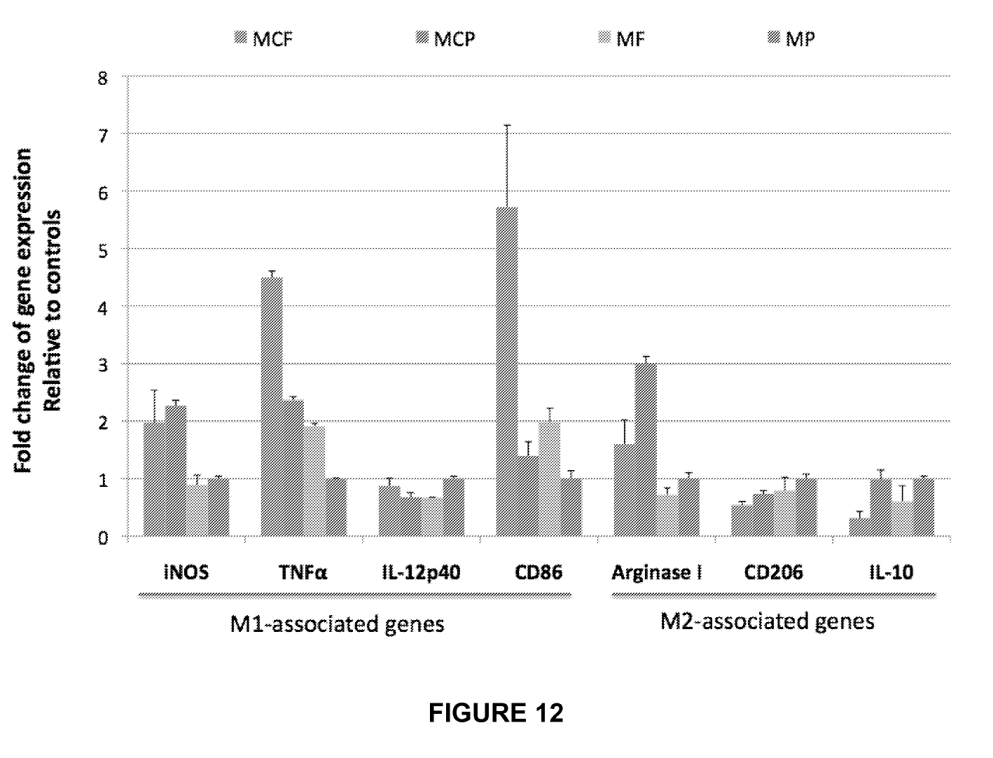

M1/M2-associated gene expression profiles were measured using quantitative real-time PCR to assess whether USPIO modifies macrophage polarization. Results showed that a 12 hour incubation of bone marrow derived macrophages with ferumoxytol (2.73 mg/mL) significantly upregulated macrophage TNFα and CD86 gene expression profiles, with or without presence of cancer cells (p<0.05, FIG. 12). Cancer cells alone induced a mild activation of TNFα (p<0.05) and Arginase I expression (p<0.05), but not CD86 (p>0.05). Ferumoxytol downregulated Arginase I, IL-10 and CD206 gene expressions in macrophages, in the presence and absence of cancer cells (p<0.05). We did not detect an induction of IL-12p40 expression, instead, we found a reduction of IL-12p40 gene expression in macrophage cells coincubated with either cancer cells, ferumoxytol, or both (p<0.05). In regards to iNOS gene expression, ferumoxytol alone did not increase iNOS expression (p>0.05). However, addition of cancers cells caused a significant induction of macrophage iNOS expression (p<0.05).

### CONCLUSION

Our data demonstrate an immunotherapeutic effect of iron oxide nanoparticles against cancer cells. Iron oxide nanoparticles can illicit a pro-inflammatory immune response in (early) cancers, which leads to polarization of incoming macrophages to M1 phenotypes, which exert a cytotoxic effect against cancer cells.

Due to the lack of any concomitant local or systemic toxic side effects, this approach might be useful to inhibit local tumor recurrence during the gap between surgery and start of adjuvant chemo-radiation. Since the iron oxide ferumoxytol is FDA-approved for intravenous treatment of iron deficiency and, therefore can be readily applied clinically via an “off label” use, our approach would be readily applicable in cancer patients.

Although the examples herein showed the delivery of ferromagnetic nanoparticles by injection into a tumor mass, other modes of delivery may also prove efficacious. For example, delivery to the tumor mass through the tumor vasculature by injecting the pharmaceutical composition into a blood vessel leading into the tumor mass, or by intravenous delivery to a site removed from the immediate vicinity of the targeted tumor.

Although the examples herein showed results of Ferumoxytol, it is considered that other ferromagnetic particles could be usefully be employed for the same purpose, such as, but not limited to, ferumoxides (Endorem/Feridex), ferumoxtran-10 (Sincerem/Combidex), feruglose (Clariscan), ferucarbotran (Resovist), ferucarbotran (Resovist S), GEH121333 and P904 and its derivatives.

Furthermore, coating of the nanoparticles provides opportunities for conjugating targeting ligands specific for directing the ferromagnetic nanoparticles to a particular type of cancer cell or tumor.

What is claimed is:

1. An immuno-therapy for treatment of a tumor, comprising:
(a) administering in vivo and to cancer cells of said tumor an effective dose of a pharmaceutically accepted composition, wherein said composition comprises superparamagnetic iron oxide nanoparticles;
(b) once said composition has been administered avoiding direct cytotoxic effects on said cancer cells and normal or healthy tissue as a result of: (i) heat applied to said composition-administered cancer cells, (ii) irradiation energy applied to said composition-administered cancer cells, (iii) a release of a toxic agent by said administered composition or to said administered composition, or (iv) any combination thereof;
(c) imaging said tumor during said immuno-therapy to determine a size of said tumor; and
(d) imaging said composition-administered to cancer cells during said immuno-therapy to determine an amount of said composition remaining in an enviroment of said cancer cells,
wherein both of said imaging steps do not cause said direct cytotoxic effects on said cancer cells and normal or healthy tissue.

2. The method of claim 1, wherein said superparamagnetic iron oxide nanoparticles comprise ferumoxytol, ferumoxtran-10 or ferumoxides.

3. The method of claim 1, wherein said effective dose comprises: (i) 1-50 mg Fe/kg body weight or (ii) 1-10 mg Fe/ml of an administered iron product concentration.

4. The method as set forth in claim 1, further comprising administering in vivo to said cancer cells of said tumor or remaining cancer cells of said tumor an additional effective dose of said pharmaceutically accepted composition when it is determined that: (i) said tumor starts to grow, (ii) said remaining composition falls below a threshold, or (iii) a combination thereof.

5. The method of claim 4, wherein said additional effective dose comprises: (i) 1-50 mg Fe/kg body weight or (ii) 1-10 mg Fe/ml of an additional administered iron product concentration.

6. The method of claim 1, wherein said composition has been chemically modified to attract or activate immune cells.

7. The method of claim 1, wherein said immune cells are macrophages or T-cells.

8. The method of claim 1, wherein said imaging in steps 1(c) and 1(d) is Magnetic Resonance Imaging (MRI).

9. The method of claim 1, wherein said imaging in steps 1(c) and 1(d) is performed as a single imaging procedure or as different imaging procedures.

10. An immuno-therapy for treatment of a tumor, comprising:
(a) administering in vivo and to cancer cells of said tumor an effective dose of a pharmaceutically accepted composition, wherein said composition comprises superparamagnetic iron oxide nanoparticles, and wherein said effective dose comprises: (i) 1-50 mg Fe/kg body weight or (ii) 1-10 mg Fe/ml of an administered iron product concentration;
(b) imaging of said tumor during said immuno-therapy to determine a size of said tumor; and
(c) imaging of said composition-administered to cancer cells during said immuno-therapy to determine an amount of said composition remaining in an enviroment of said cancer cells.

11. The method of claim 10, wherein said superparamagnetic iron oxide nanoparticles comprise ferumoxytol, ferumoxtran-10 or ferumoxides.

12. The method as set forth in claim 10, further comprising administering in vivo to said cancer cells of said tumor or remaining cancer cells of said tumor an additional effective dose of said pharmaceutically accepted composition when it is determined that: (i) said tumor starts to grow, (ii) said remaining composition falls below a threshold, or (iii) a combination thereof.

13. The method of claim 12, wherein said additional effective dose comprises: (i) 1-50 mg Fe/kg body weight or (ii) 1-10 mg Fe/ml of an additional administered iron product concentration.

14. The method of claim 10, wherein said imaging in steps 1(b) and 1(c) is Magnetic Resonance Imaging (MRI).

15. The method of claim 10, wherein said imaging in steps 1(b) and 1(c) is performed as a single imaging procedure or as different imaging procedures.

16. The method of claim 10, wherein said composition has been chemically modified to attract or activate immune cells.

17. The method of claim 10, wherein said immune cells are macrophages or T-cells.

18. The method of claim 10, further comprising, once said composition has been administered, avoiding direct cytotoxic effects on said cancer cells and normal or healthy tissue as a result of: (i) heat applied to said composition-administered cancer cells, (ii) irradiation energy applied to said composition-administered cancer cells, (iii) a release of a toxic agent by said administered composition or to said administered composition, or (iv) any combination thereof.
